(19) United States
(12) Patent Application Publication
(10) Pub. No.: US 2017/0157410 A1
(43) Pub. Date: Jun. 8, 2017

(54) SYSTEMS AND METHODS FOR SHARING THERAPY PARADIGMS IN A NEUROMODULATION SYSTEM

(71) Applicant: BOSTON SCIENTIFIC NEUROMODULATION CORPORATION, Valencia, CA (US)

(72) Inventors: Michael A. Moffitt, Saugus, CA (US); Jordi Parramon, Valencia, CA (US); Goran N. Marnfeldt, Valencia, CA (US)

(73) Assignee: BOSTON SCIENTIFIC NEUROMODULATION CORPORATION, Valencia, CA (US)

(21) Appl. No.: 15/367,515

(22) Filed: Dec. 2, 2016

Related U.S. Application Data
(60) Provisional application No. 62/263,084, filed on Dec. 4, 2015.

(51) Int. Cl. A61N 1/372; G06F 19/00; H04L 29/06; A61N 1/36

(52) U.S. Cl. CPC A61N 1/37247; A61N 1/3605; G06F 19/3481; G06F 19/3418; H04L 63/102; G06F 19/324

(57) ABSTRACT

Methods, devices and systems for sharing therapy patterns for treating neurological disorders. A developed therapy pattern may be shared from one physician to another in several ways. In one example, a device provides to a physician one or more of a pseudocode for a therapy or an optical machine readable representation of a therapy pattern which can be shared via text, photo, email, etc. In another example, a physician may upload a therapy package comprising a therapy pattern and related information to a repository or library and can be provided a code or name to share, such that another physician may download the therapy pattern using the shared code or name. In yet another example, a searchable repository or library is provided. Devices or systems may provide safety and integrity checks on uploaded or downloaded patterns, and may limit which persons may upload or download.

### CROSS REFERENCE TO RELATED APPLICATIONS

The present application claims the benefit of and priority to U.S. Provisional Patent Application Ser. No. 62/263,084, filed on Dec. 4, 2015, the disclosure of which is incorporated herein by reference.

### BACKGROUND

Implantable and/or wearable stimulations systems for the treatment of various diseases and disorders of the neurological system have proven effective in a wide variety of ways. For example, spinal cord stimulation (SCS) systems are accepted treatments for chronic pain syndromes. Deep brain stimulation (DBS) systems have been used for chronic pain as well, and are gaining acceptance for treatment of movement and tremor disorders. Peripheral nerve stimulation (PNS) systems have also been shown effective for certain indications, and functional electrical stimulation (FES) has been investigated for restoration of functionality to paralyzed extremities. These and other therapies are under investigation for numerous indications beyond those already in use.

Historically, available systems facilitated a limited variety of therapeutic output waveforms, such as voltage or current controlled square waves. A proposed new hardware and/or embedded software arrangement will remove some of the existing limitations on waveform type. Newly developed waveforms may be significantly more complex than those previously in use. Therefore it is desirable to identify and develop new approaches to facilitate sharing newly developed therapy patterns.

### OVERVIEW

Methods, devices and systems for sharing therapy patterns for treating neurological disorders. A developed therapy pattern may be shared from one physician to another in several ways. In one example, a device provides to a physician one or more of a pseudocode for a therapy or an optical machine readable representation of a therapy pattern which can be shared via text, photo, email, etc. In another example, a physician may upload a therapy pattern to a repository or library for therapy patterns and can be provided a code or name to share, such that another physician may download the therapy pattern using the shared code or name. In yet another example, a searchable repository or library is provided. Devices or systems may provide safety and integrity checks on uploaded or downloaded patterns, and may the persons who can upload or download. Compensation schemes for physicians who share therapy patterns may also be offered.

This overview is intended to briefly introduce the subject matter of the present patent application, and is not intended to provide an exclusive or exhaustive explanation of the invention. The detailed description is included to provide further information.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows various functional components of an illustrative neurostimulation system;

FIG. 2 shows an implantable stimulator and electrodes;

FIG. 3 shows an illustrative deep brain stimulation system as implanted;

FIG. 4 shows an illustrative spinal cord stimulation system as implanted;

FIG. 5 shows an output architecture for an illustrative pulse generator;

FIGS. 6-11 show several illustrative therapy patterns;

FIGS. 12 and 13 show how a therapy pattern may be stored and/or executed;

FIG. 14 shows an illustrative method in block flow form;

FIG. 15 shows an example data structure for a therapy script;

FIGS. 16-18 show illustrative methods in block flow form;

FIG. 19 shows a screen shot for creation of a therapy package;

FIG. 20 shows an illustrative method in block flow form;

FIG. 21 shows a screen shot for an illustrative therapy pattern search interface; and

FIGS. 22-24 show illustrative embodiments in schematic form; and

### DETAILED DESCRIPTION

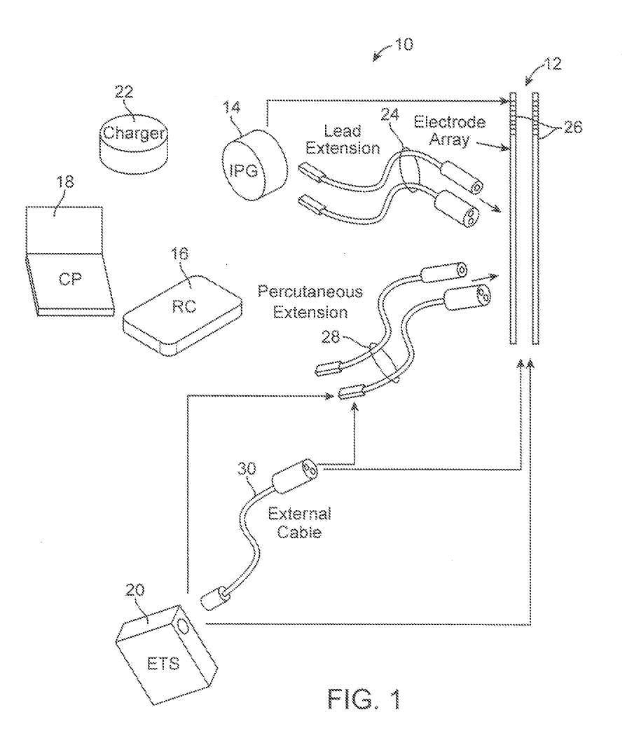

FIG. 1 shows a system for providing neurological therapy, for example, as spinal cord stimulation (SCS), deep brain stimulation (DBS), peripheral nerve stimulation (PNS), or functional electrical stimulation (FES). The system 10 includes electrodes 12 configured for coupling to an implantable pulse generator (IPG) 14. The IPG may communicate with one or more of a patient remote control (RC) 16, a clinician programmer (CP) 18, and/or a charger 22.

An external testing system (ETS) 20 may also be provided for testing therapy parameters prior to implantation of the IPG, using percutaneous extensions 28 and, as needed an external cable 30 to couple to the implantable electrodes 12. If needed, lead extensions 24 may be used to couple the IPG to the implantable electrodes 12.

As shown in FIG. 1, the implantable electrodes may include arrays of electrode contacts on linear leads 26; in other examples, paddle leads may also be used. One, two or even four leads 12 may be provided, with up to 32 total contacts available in modern systems; in the future more or fewer contacts on more or fewer leads may be provided depending the particular system.

The IPG 14 can couple directly to the electrodes 12 or may be coupled via the lead extensions 24, depending on the positioning of each element as implanted. The IPG may include a rechargeable battery and charging coil to allow recharging when placed in proximity to the charger 22. Alternatively, the IPG may use a non-rechargeable battery and omit the charging coil. In some examples, the IPG may be externally powered and omits a battery entirely.

The CP 18 can be used by a physician to manipulate the outputs of the IPG 14 and/or ETS 20. For example the CP 18 can be used by the physician to define a therapy regimen or program for application to the patient. Multiple programs may be facilitated and stored by the IPG 14 or ETS 20; in some examples, the RC 16 may store the programs to be used. Communication amongst the IPG 14, RC 16, CP 18, ETS 20 and Charger 22 may use any suitable protocol such as wireless RF telemetry, inductive communication, Bluetooth, etc.

The RC 16 may be used by a patient to enable or disable therapy programs, to select between available programs, and/or to modify the programs that are available for use. For example, in some embodiments a patient may use the RC 16 to activate a stored program and then manipulate therapy by increasing or decreasing therapy strength and/or changing therapy location, within limits set by the physician.

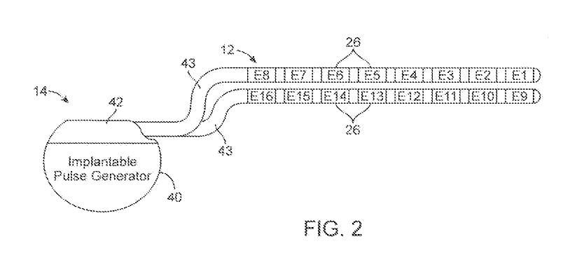

FIG. 2 shows an implantable stimulator and electrodes. As shown in the closer detail here, the IPG 14 may include a canister 40 and header 42. The canister 40 is conductive in most examples, using biocompatible materials such as titanium and/or stainless steel, for example, to allow use as an electrode when implanted. The header allows removable connection to the lead 12, which in this example may have a bifurcation or yoke allowing two segments 43 to extend therefrom, to two arrays 26 at the distal end of the lead 12. The electrode arrays 26 can be numbered as shown to facilitate ease of understanding when programming, with, for example, one array marked electrodes E1 to E8 on one of the lead segments 43, with E1 being distalmost. Other conventions may be used.

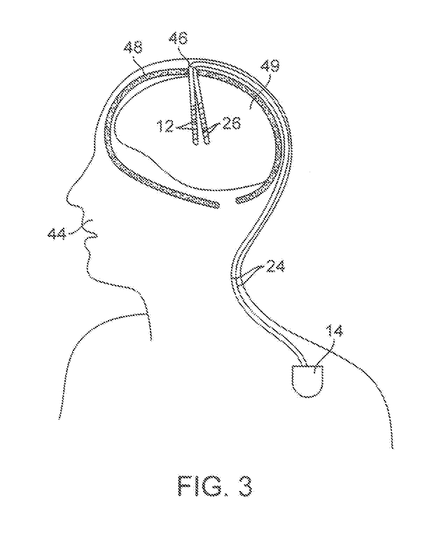

FIG. 3 shows an illustrative deep brain stimulation system as implanted. The IPG 14 may be implanted in the patient's upper chest or neck region, with a lead extension 24 going up the neck to the head. The lead 12 is inserted through an anchor 46 that can be placed in a burr hole formed through the cranium 48 of the patient, allowing the arrays 26 at the distal end of the lead 12 to be placed inside the brain 49. The procedure can be performed using known visualization techniques and technologies so that the electrodes on lead 12 may be placed in proximity to a desired structure for therapy. Other locations the IPG 14 and/or lead 12 may be used.

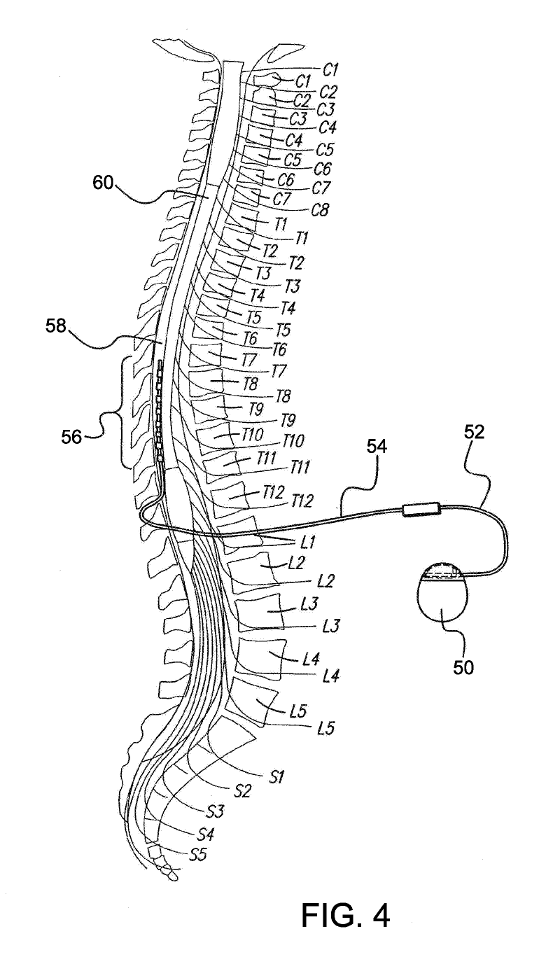

FIG. 4 shows an illustrative spinal cord stimulation system as implanted. In this example, an IPG 50 may be placed near the buttocks or in the abdomen of the patient, with or without a lead extension 52 for coupling to the lead(s) 54 that enter the spinal column. Region 56, at about the level of the lower thoracic or upper lumbar vertebrae may serve as an entry point to the spinal column, where the distal end of the lead 54 with an electrode array may be placed close to the spinal cord 58. Other locations for the IPG 50 and/or lead 54 may be used.

The standard approach to therapy in systems similar to those shown in FIGS. 1-4 has been that the IPG 14 (and ETS 20) may offer current controlled or voltage controlled therapy comprising either biphasic square waves or monophasic square waves having passive recovery. In general, the amount of current out of an electrode should zero out over time to avoid encouraging corrosion at the electrode-tissue interface. For this reason, biphasic pulses, or monophasic pulses with a passive recovery period are typically used.

An individual component of a therapy program, in these systems, controls a subsequent component or impulse. For example, when delivering a biphasic square wave therapy, the duration and amplitude of the first phase controls the duration and amplitude of the second phase, in order to achieve charge balancing. In a monophasic therapy with passive recovery, again, the duration and amplitude of the active phase ultimately determines the length and strength of the recovery signal applied thereafter. Such an approach may be integrated in the hardware and/or software design as a set of rules for therapy delivery.

In some examples, the present invention does away with this limiting approach to therapy delivery by allowing arbitrary functions to be achieved within a therapy program, while applying rules for charge balancing, duty cycle and the like, across the program rather than on a pulse-to-pulse basis. With greater flexibility, a physician will be able to use the system to accomplish more varied waveforms. As a result, the physician is allowed to take further steps in the development of new therapy waveforms for new therapy indications. Much of the following discussion focuses on how physician may use this new capability.

More recent launches by some companies, and ideas still in development, include the concepts of burst stimulation and high frequency stimulation. Burst stimulation is merely the provision of a concatenation of biphasic pulses end to end, provided in short “bursts” at intervals. For example, burst therapy may deliver 500 Hz square waves may be delivered for 5 cycles, with the sets of 5 cycles repeated every 25 milliseconds (40 Hz). High frequency stimulation, for example, at 10 kHz, is simply the same therapy offered at high rates. While these options are available using the flexible architecture described herein, neither is as flexible as the therapy described and shown herein. The wider variety of variables may facilitate therapy development paradigms that are further described below.

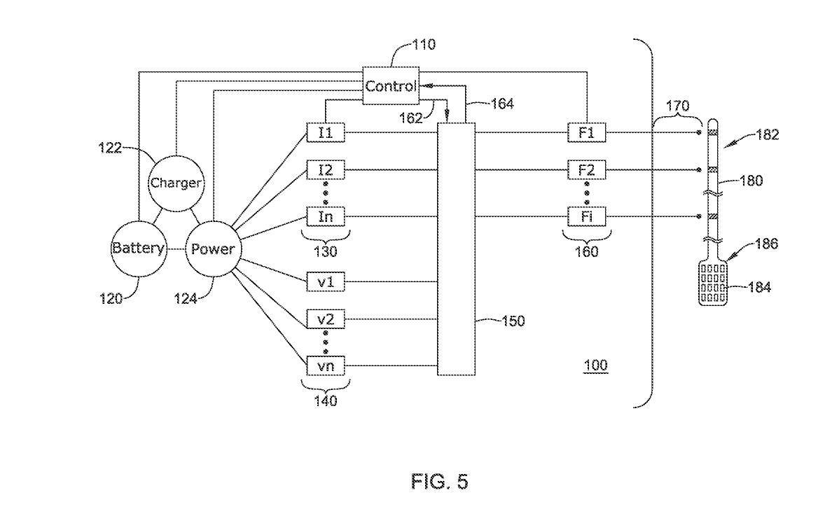

FIG. 5 shows an output architecture for an illustrative pulse generator, which may be implantable or external. The device 100 includes a control block 110. The control block 110 may be implemented as a microcontroller or microprocessor, with associated memory banks to store instruction sets to be implemented as appropriate/needed, and/or data for later recall. In other examples, the control block 110 may be implemented as a state machine, or as a combination of circuits including, for example, various logic and memory circuits and analog, mixed signal, or digital application specific integrated circuit (ASIC) components. The control block may include suitable analog to digital conversion circuitry and, if desired, digital signal processing circuits. Though not shown the device 100 may be include telemetry and other circuitry to perform various well known functions such as communicating to an external programmer and/or remote control.

A battery 120 may be used to provide power to the system, and may be coupled to a charger 122 if the battery is rechargeable. In some examples, a non-rechargeable battery 120 is used and the charger 122 can be omitted. As another alternative, the battery 120 may be omitted and the system may be operable when the charger 122 receives external power.

A power unit 124 is also shown. The power unit 124 may provide various power outputs to support therapy driving circuitry that can include a plurality of current controlled sources 130 and/or voltage controlled sources 140. Single or plural sources 130/140 may be provided, and any number of each can be used. In other examples, the sources may be convertible between voltage or current supply. The sources 130/140 may be fixed or variable. To support a variety of sources, the power unit 124 may have voltage controlled output lines, for example, 3, 5 or 15 volt supply lines (or other voltage level), and/or one or more compliance voltage sources (using for example a capacitor coupled to a voltage multiplier or booster) that maintains adequate headroom to drive the current sources 130. Various implementations for each of elements 120, 122, 124, 130 and 140 can be used.

An output controller 150 couples the sources 130/140 to output filters 160 and contacts 170 for coupling to a lead 180 or lead extender (not shown). The output controller 150 may simply connect a dedicated source or sources 130/140 to a single output 170 via hardwire or via switches, or, in other implementations, may multiplex the various sources 130/140 to various outputs by including a multiplexor or switch array.

The plurality of filters 160 may be dedicated to each of the outputs 170, as shown, or may instead be switchable in and out of association with the individual outputs 170, or may be omitted entirely depending on the nature of the output controller 150 and/or sources 130/140. The microprocessor 110 may control the filters 160, as shown, as well as the sources 130/140 and output controller 150. If sensing capability (sometimes referred to in particular for neurological therapy including neurostimulation or neuromodulation as closed loop sensing) is provided, the microprocessor 110 may not only have a control line 162 to the output controller 150, but may receive signals on a sensing line 164. The sensing line 164 may instead be linked directly to the contacts 170.

The contacts 170 may be provided in a header (not shown) for coupling to the lead 180. One or more contacts 170 may couple to the housing of the device 100 which may serve as one or more electrodes for use in the patient. For example, button electrodes may be provided on the device 100 and/or a large portion of the device housing may serve as a single large electrode.

The lead 180 includes a plurality of spaced apart contacts 182 for coupling to contacts 170. In this example, the lead is shown as having electrodes 184 at its distal end arrayed on a paddle 186. A plurality of leads 180 may be used. The lead 180 may instead be coupled to the device 100 using a lead extension. The lead 180 may also take the form of a split lead having a yoke and multiple paddles 186 or other distal structures having electrodes thereon. In one example, rather than a lead, an ultrasound, RF or other energy output may be provide to activate remotely located electrodes.

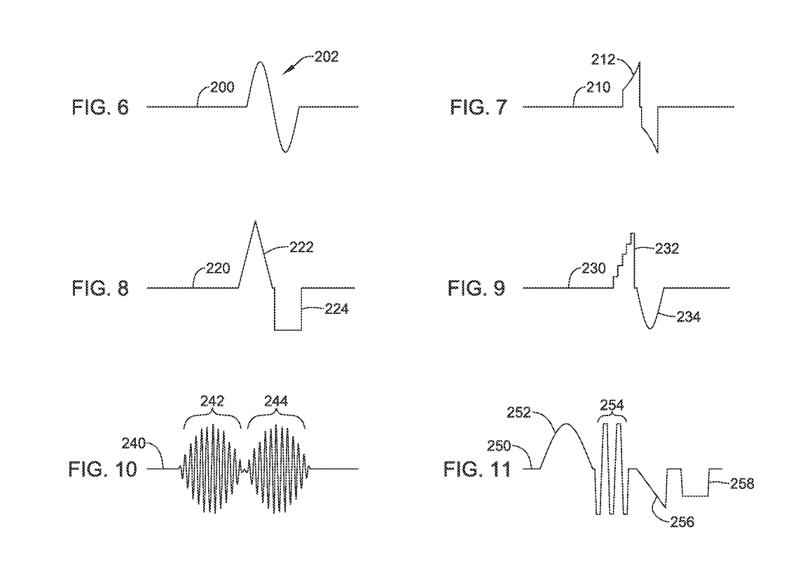

FIGS. 6-11 show several illustrative therapy patterns. Historically, a square wave output would be provided by closing a switch or turning on a given output at a specific level; for a more complex, non-square wave output, other approaches may be taken. It should be noted that the signals shown may be configured as voltage controlled or current controlled outputs. In either case, a digitized output may provide sequential individual outputs at given levels to approximate a curve. Alternatively, for a controlled current curve, a current mirror may be coupled to analog circuitry that provides a controlled voltage across a resistor, generating a current output through the resistor which can then be copied using the current mirror to develop a controlled current output. For a controlled voltage curve, an analog circuit may be configured to generate a desired curve and a resultant voltage passed through a buffering amplifier (or gain amplifier) to provide the output.

FIG. 6 shows, as an example, a single cycle of a sinusoidal therapy pattern. Output 200 remains at essentially zero until the sinusoid is delivered at 202. In one example, the sinusoid 202 is made up of a series of individual steps and is basically a digital approximation. In another example, one or more of the sources within a device may be configured as an analog sinusoid (for example, a driven RC circuit having a controllable frequency by virtue of making the resistor or capacitor of the RC circuit variable, coupled to a buffer/amplifier), and the output is switched on for the analog sinusoid.

FIG. 7 shows another example in which an up-ramped exponential function is provided, with the signal 210 at zero until the function 212 is applied. FIG. 8 shows an output 220 with up and down ramps (a triangle wave) at 222, followed by a square wave 224. FIG. 9 shows an output 230 with a stepped output 232 followed by a sinusoidal half-cycle 234. FIG. 10 shows an amplitude modulated carrier signal with first cycle 242 and second cycle 244.

FIG. 11 shows a longer script in which an output 250 includes a sinusoidal half wave 252, followed by a short burst 254, a ramp 256 and a square wave 258. Other combinations may be provided, with different components of the waveform having different internal purposes. For example, a high frequency burst 254 in FIG. 11 may serve a purpose of minimizing the sensation of paresthesia (the tingling sensation associated with standard SCS) to a patient, while a square wave 258 may be provided to block a pain signal.

Assuming delivery by a single pair of electrodes, each of FIGS. 6-11 are shown with approximately balanced anodic and cathodic outputs; this need not be the case in all examples but is likely typical. However, different elements of the pattern shown in FIG. 11 may be generated across different electrode combinations. For example, assuming delivery by a system as shown in FIG. 2, the sinusoidal half wave of FIG. 11 may be delivered via a selected pair of electrodes 26 (for example, E11 as anode and E2 as cathode), followed by the high frequency burst 254 via a different selected pair of electrodes 26 (for example, between E10 and E7), with the ramp 256 delivered via yet another pair (E3 and E4), and the square wave 258 delivered across a different combination (E2 and E4 as anodes, and E11 as cathode).

As more complex patterns develop, several rules for charge balancing electrode interfaces may also be developed to ensure that short term, long term, and intermediate term rules to avoid encouraging electrode interface degradation/corrosion are avoided. For example, a first rule may be that the long term, average charge on an interface be zero—thus, over a period in the range of about ten minutes to twenty-four hours, the amount of current through a single interface should balance out to zero. A second rule may be that the mid-term period in the range of about one second to about ten minutes, average charge on an electrode interface of a given size and material be less than a preset quantity (i.e., no more than 10 milli-coulombs), which may be determined by tracking current and time period for each electrode interface. Finally, short term rules may call for a maximum quantity of current in the near term, such as less than 10 seconds, and/or a maximum instantaneous current or voltage.

While the short term rules and long term rules may already be understood for existing systems with limited capabilities, the intermediate rules become more relevant with more complex waveforms and patterns. Intermediate term rules may also take the form of a curve or set of curves, for example, a maximum charge burden may be defined as the amount of charge held over time on an interface, and may be subject to a set of rules for different time periods. In an example, a maximum quantity of charge to hold may be defined for periods of one second, five seconds, and ten seconds, with the maximum charge decreasing as the period of time increases.

As a result of such rulesets, a pattern as shown in FIG. 11 and described above may include a first portion, as shown, which is the therapeutic design, and a second portion applicable across the various electrodes to address intermediate term and long term rules, with the second portion designed to apply at sub-threshold levels to avoid paresthesia, for example, or other stimulus, while balancing out charge on the electrode interfaces.

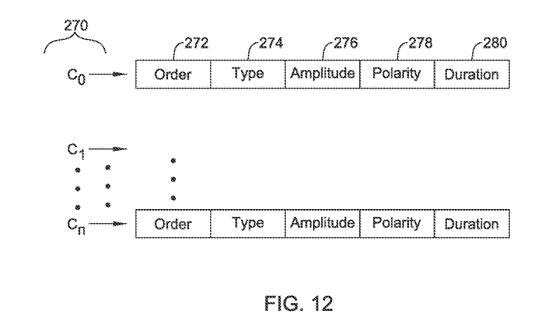

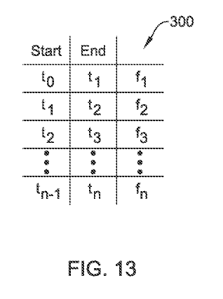

FIGS. 12 and 13 show how a therapy pattern may be stored and/or executed. Referring first to FIG. 12, a therapy may be provided essentially as a script of sequential commands. The commands may be numbered as shown at 270 [C₀ . . . Cₙ], for example. Each command can include an indication of its order 272 within a sequence. The command can include an indication of type 272 which may determine whether a voltage controlled output or a current controlled output is to be provided. Type 272 may also indicate which electrodes are to be used in delivering the output. Type 272 may include a shape determination as well, such as whether a fixed, ramped, curved, sinusoidal, or exponential output is to be generated. The peak amplitude 276 may be indicated, as is polarity 278 (which may instead by a subcomponent of type, if desired), and duration 280. As a group these elements may be stored as a memory structure or object. A detailed example of storing of a therapy pattern may be found as well in U.S. Pat. No. 9,144,687, the disclosure of which is incorporated herein by reference.

For example, an approximation of a sinusoid may be provided as a series of individual outputs with varying amplitude 276 that rises, falls, and rises again, to mimic the curvature of the sinusoid. In another example, an exponential output function may be designated with start and stop points and a curve definition indicating, for example, the time constant of the exponential. In another example, a sinusoidal function may be provided by a single element, for example, C₀ may comprise a type element 274 indicating the frequency of the sinusoid, the duration 280 indicating the number of cycles, or fraction of a single cycle to be implemented, polarity 278 indicating the leading polarity, and the amplitude 276 may define the peak amplitude.

FIG. 13 shows another manner of representing a therapy pattern. In this example, a given device may make available a function set that can be called, with each function set reliant on one or more parameters. In the example, a table 300 represents a therapy pattern having an overall duration of to less to, in which functions f₁ to f₀ are used for durations defined by intermediate time points t₁ . . . tₙ₋₁. The individual functions may be data types defining sets of variables. For example, if function f₁ is a square wave, it may require inputs of amplitude, duration, and polarity. If function f₂ is a ramp, it may require inputs of starting amplitude, end amplitude, duration, and polarity. If function f₃ is a sinusoid, it may require inputs of frequency, amplitude, leading polarity, and cycle fraction/quantity. Other or additional functions may be provided. A null function may be provided, and functions may be callable in voltage or current modes. The set of functions is called in an order and for durations defined by the table 300. A more complex pattern may use overlapping functions operable on different sets of electrodes such that, for example, function f₁ is applied by a first electrode set, and function f₂ is applied via a second electrode set, with at least a portion of the two functions overlapping.

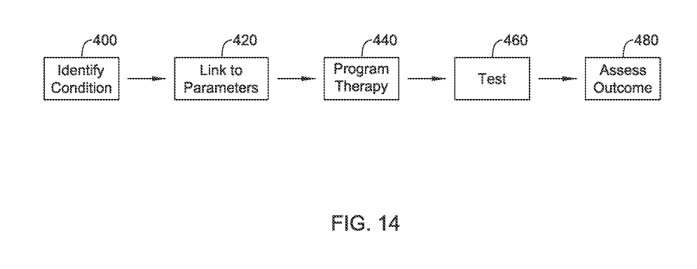

FIG. 14 shows an illustrative method in block flow form. The purpose in FIG. 14 is to plot out a manner in which a physician may develop a new therapy pattern to treat a given patient condition. A physician may identify the condition of the patient needing treatment, as indicated at 400, and then link the condition to therapy parameters, as indicated at 420. This linkage can be established using various approaches.

For example, in order to excite a given cell type in the patient's neurological system, a minimum electric field may be required, such that once a particular cell type is known as responsible for a given condition or susceptible to therapy to treat, alleviate or resolve the given condition, measuring (in vivo or in vitro, for example), or determining through analysis (using known qualities of the cell such as response to a hormone, drug, chemical or biologic substance, or knowing the receptors of a cell such as calcium or potassium channel receptors) a known field level that can cause a response, will provide a link between a parameter and the identified condition.

In another example, the parameter link 420 may also include an understanding of secondary responses. For example, simply knowing what field level may excite a given cell to cause that cell to become refractory at a desired time, or to generate an output signal for transmission, is sometimes only the first piece of the linkage at 420. In an embodiment, after delivering a primary field needed to achieve a desired response from a targeted cell, a second therapy output may be generated to minimize side effects of the primary field. This may include, for example, mixing two types of therapy. A current controlled impulse may be used to target a desired cell structure at a desired location—since current controlled outputs can facilitate narrowly tailored fields—and may be followed and/or preceded by a voltage controlled output to place a larger, and less specific region of nerve tissue into a desired state.

In one such example, referring to FIG. 2, electrodes E2 and E10 may be used as cathodes and electrodes E4 and E12 as anodes for a single polarity voltage output pulse, followed by a current controlled biphasic output using electrode E3 as anode and electrode E11 as cathode in the leading phase, followed by a recovery outputs using E2 and E10 as anodes and electrodes E4 and E12 as cathodes for a single polarity output pulse. The first voltage controlled output pulse in such a sequence may be used to place a volume of tissue in a first state, such as a refractory state, the current controlled biphasic output may be used to stimulate a targeted region of neural tissue, and the second voltage controlled output may be used to balance out the charge delivered. Other sequences may be used instead.

In another example, a physician or researcher may engage in an iterative testing process in which a therapy's primary purpose may be gleaned and addressed in a first pass, developing the therapy itself, and then testing various approaches to reducing side effects with subsequent testing. For example, a first testing may be done to prove that the original problem for which therapy is applied can be addressed, thus, in a hypothetical, a patient's pain sensation may be first addressed with therapy delivery, causing side effects such as paresthesia. Multiple secondary therapies may then be applied to determine effects on the paresthesia in order to reduce or eliminate it.

In another example, a primary therapy may be developed to identify parameters, with additional pre-therapy testing performed, for example, to determine ways to optimize the effect of the therapy to be delivered. In an example, an impulse is determined to address a movement disorder or tremor, and pre-pulsing may be tested to determine whether the impulse amplitude may be lowered, for example, to reduce power consumption or side effects. A number of additional illustrations are outlined below.

Once a parameters link is identified at 420, therapy can be programmed 440. Therapy programming may be automated or manual and can take several forms. For example, a system may be designed to provide a physician a visualization of anatomical structures in a patient relative to implanted electrodes—or yet-to-be-implanted electrodes—and the physician identifies an anatomical location and designates a desired field level, from which the system determines an appropriate electrode combination to generate the desired field. An iterative process may follow in which the physician then reviews one or more proposed therapies that would generate the desired field, and could select one or several proposed therapies to test and then set about designating secondary therapies to mitigate against side effects of the proposed therapies.

In an alternative, the physician may use the CP to write the details of a given therapy pattern. This may include, for example, programming individual steps within a therapy pattern, and/or selecting functions to perform within a pattern. In another alternative, a physician may use a separate computer to write a therapy pattern and, transfer the therapy pattern using a transferrable memory (SD card or thumb drive for example), Bluetooth, WIFI or other communications to the CP or other in-system device such as the ETS, RC or IPG.

With a therapy pattern programmed at 440, therapy is tested at 460, and the outcome is then assessed at 480. Therapy testing 460 may include one or many subjects. Outcome assessment 480 may include direct observation of the therapy recipient, query and answer with a participant, and/or the use of imaging or measurement electronics to determine how particular anatomical structures respond. For example, evoked signals from neurons can be measured, or the strength of muscle contraction, extent of muscle relaxation, changes in other observables such as brain activity, tremor, memory, and cognition, etc. may be monitored and quantified.

U.S. Provisional Patent Application No. 62/263,073, titled SYSTEMS AND METHODS FOR THE DEVELOPMENT OF THERAPY PARADIGMS FOR NEUROLOGICAL TREATMENTS, filed on Dec. 4, 2015, provides several examples in which new therapy protocols and patterns can be developed for identified conditions, and is incorporated herein by reference.

As illustrated above in FIGS. 6-11, a wide variety of waveform patterns may be output by a device as described herein. This may make it more difficult for one physician to communicate to another about the type of waveform that is being applied to treat a specific patient condition. For example, a set of programs for a given patient may be as follows:
Program 1—“Lower Back”, Amplitude 1.0 V, Pulse Width 220 μs, Rate 400 Hz, electrodes 14 and 16 anodes, electrode 7 cathode.
Program 2—“Leg”, Amplitude 2.4 V, Pulse Width 100 μs, Rate 80 Hz, electrode 11 anode, electrode 3 cathode.
   For a physician to share either program of this sort with another physician is relatively easy; indeed, 2-3 lines of text is all that would be needed. However, to communicate a description of a waveform as shown above in FIG. 11, may be more complex:
Program N—“Complex Pattern”, half-sine wave at 400 Hz with amplitude V1, 2.5 cycles of square wave at 2400 Hz with amplitude 1.1*V1, negative ramp from 0 v to −0.8*V1 lasting 1.25 ms, followed by half square wave at −0.5*V1 with duration 900 μs.
   Where V1 is a reference amplitude for the overall pattern. Program N is not readily conveyed. Instead, communication may take several back and forth discussions and cross-checks to make sure that all information is correctly provided. Moreover, determining that a program has been incompletely or incorrectly communicated is rendered difficult insofar as not all patients respond to all therapies, different patients may require different threshold voltage/current/field levels, and some conditions do not demonstrate immediate response to therapy itself. Further adding to the complexity of communicating patterns for use is that durations of time allocated to charge recovery (including active or passive recovery) may be assigned as part of the pattern as well.

In a first illustrative example, rather than reciting the individual elements of a therapy pattern in a textual, descriptive manner, a tool for condensing a complex pattern into a text/data string that is readily communicated is provided. The condenser tool may be a software function that can be called by a programmer (such as a CP), or it may be an application that can be loaded onto a mobile device (such as a tablet or smartphone), or it may be an online tool provided for access by physicians or researchers working on therapy pattern development who desire to share patterns.

FIG. 12 is useful in this context. When converting a set of instructions, the order 272 may (optionally) be stripped out, as order can be represented by the order of ASCII (or other) characters output by the condenser tool. Next, the type may be reduced to a single bit (0/1) representing either current control or voltage controlled output. The amplitude 276 can be a relative term relying on a % of a reference amplitude, which may be the maximum voltage of a given device, or may be variable for the waveform pattern. Thus, amplitude 276 could be stored, depending on desired resolution, as 6 bits with 11 1111 representing 100% of a reference amplitude for a given waveform, and 00 0001 representing 1/64^{th} of the reference amplitude. Next polarity and/or electrode selection 278 may be 8 bits, with the first four bits representing the set of electrodes to be chosen as anodes, and the last four bits representing the set of electrodes to be used as cathodes, for example, in a system having 16 electrodes. As would be clear to the skilled artisan, a 32 electrode system could use 10 bits instead. Duration could be indicated as a number of minimum time duration steps; for example, in a system with a pulse width maximum limit of 5 milliseconds, and steps of 10 microseconds (approximately), nine bits would give the desired resolution (512 steps).

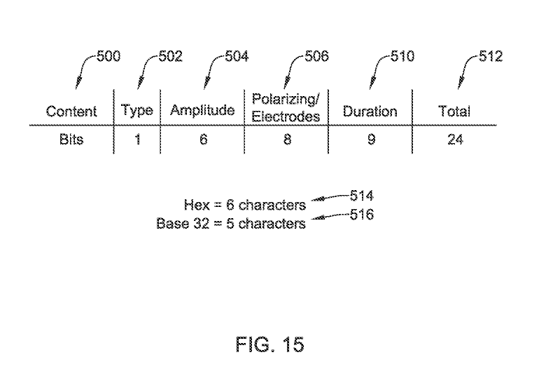

FIG. 15 illustrates the data structure 500 of an illustrative example using a therapy pattern as in FIG. 12. In the example, type 502 uses one bit, amplitude 504 uses six bits, polarity/electrode selection 506 uses eight to ten bits, and duration 510 uses nine bits. A total 512 of twenty-four to twenty-six bits would represent a given step in a signal, allowing representation as 6 hexadecimal characters (0-9 and A-F) as noted at 514. As an alternative noted at 516, 5 characters in base 32 would work as well, where base 32 could readily be provided as a simple text string consisting of numbers 2 to 9 and letters A to Z, less the potentially confusing letters I and O and numbers 1 and 0. Alternatively, rather than a text string, an optical machine readable representation of data (such as a linear or matrix bar code) may be used instead.

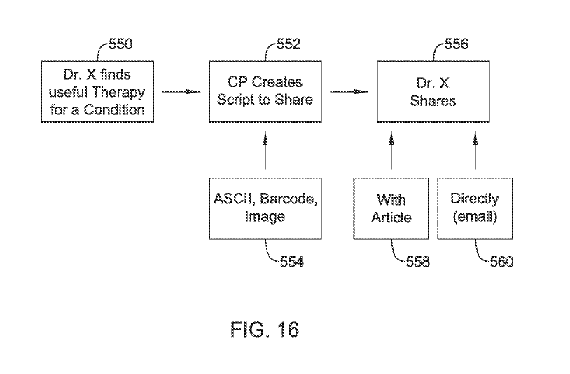

FIG. 16 shows an illustrative example. A user, “Dr. X,” finds a useful therapy for a given condition, as indicated at 550. Dr. X may use methods and/or systems shown U.S. Provisional Patent Application No. 62/263,073, titled SYSTEMS AND METHODS FOR THE DEVELOPMENT OF THERAPY PARADIGMS FOR NEUROLOGICAL TREATMENTS, filed on Dec. 4, 2015, for example, to identify a link between a given condition and the useful therapy. A clinician programmer (CP), or an app, or website, or other condenser tool creates a shareable script, as indicated at 552. The shareable script may be, for example, in ASCII code, or an optical machine readable representation of data. Dr. X can then share the script at 556 by providing it in a footnote to an article 558, for example, or by sharing directly via email or posting to a webpage, chat, text message, etc., as indicated at 560.

A system as in FIG. 15 could be used to create fairly simple patterns for sharing in a manner as shown in FIG. 16. However, any complexity would quickly overwhelm the user, making sharable text string codes rather unwieldy. For example, referring to FIGS. 6-11, it is readily seen that various code types may be used, rather than the simple version shown in FIG. 15. For example, the sine wave in FIG. 6 could be one data type, having variables to indicate the period of the sine wave, how many cycles (plural, whole, half, quarter or less) to deliver, the peak amplitude to use, whether current controlled or voltage controlled, and whether the wave is to be repeated and, if so, at what interval. A ramp as shown at 212 in FIG. 7 may indicate duration, start amplitude, and end amplitude, as well as repeatability and whether it is mono-phasic or biphasic as shown. For each of these a byte or more of data may be needed to indicate which electrodes to pick. An amplitude modulated sinusoid as shown in FIG. 10 may require even more data points.

In an illustrative example, the type data in FIG. 15 could include several bits to accommodate a plurality of functions as indicated at 300 in FIG. 13. For example, a callable function set may be defined to include, for example, sinusoid, ramp, decay, square wave, stepped outputs, and other shapes. Within a stored program type, nested functions may be used as well, again to potentially reduce the amount of data needed to store a given program. Each function may take one or plural variables. The elements in a stored pattern would take a form of {function type, data(1), data(2) . . . data(n)} where data(1) to data (n) would represent the information taken by the function. For example:

{repeating square; amplitude; pulsewidth; interpulse delay; number of cycles}

Such a data set could 4 to 8 bits for each of the type and data components, yielding, for example, a 40 bit data representation. Even at 40 bits, however, eight symbols in base 32 would be needed to convert to a script using the method of FIG. 15/16.

Even adding to the number of types still leaves the basic problem in that, using common keyboard characters, each individual character can only represent so many bits of data. If the purpose is to share information, it should be noted that the more keyboard characters required, the more likely an error is to occur. If parity checking or the like is added, this may help but still leaves a significant likelihood of error with any complexity.

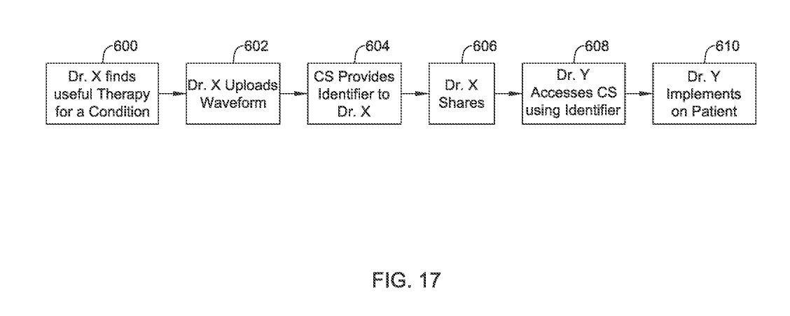

FIG. 17 illustrates another example method in block form. A user, “Dr. X,” finds a useful therapy for a given condition, as indicated at 600. Dr. X may use methods and/or systems shown in U.S. Provisional Patent Application No. 62/263,073, titled SYSTEMS AND METHODS FOR THE DEVELOPMENT OF THERAPY PARADIGMS FOR NEUROLOGICAL TREATMENTS, filed on Dec. 4, 2015, for example, to identify a link between a given condition and the useful therapy.

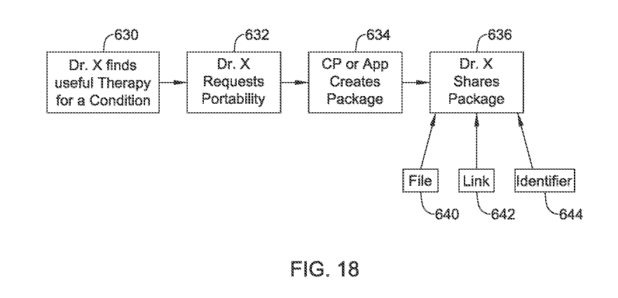

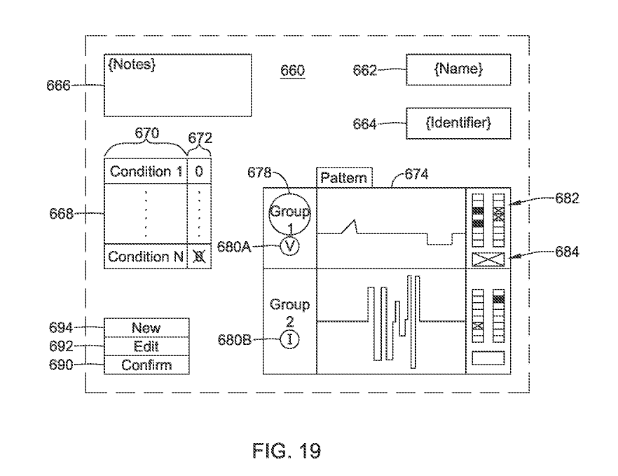

Dr. X then uploads a waveform, as indicated at 602. The uploading step may be as simple as selecting an option or pressing a button, and may be performed by one or more of the CP, RC, or via an application operating on a computer or mobile device of Dr. X. Uploading may first include the generation of a therapy package using methods such as shown in FIGS. 18-19, below, and described in associated text.

The upload at 602 may be to a central server (CS) which may be operated by or at the request or direction of a medical device manufacturer, or a university, hospital network or clinical practice to which Dr. X belongs, for example. The CS then provides an identifier to Dr. X which Dr. X can then share at 606. In this example, the CS maintains the therapy package or therapy pattern securely and provides the identifier, rather than attempting to condense the data associated with a therapy pattern into a script as described above, for example, with reference to FIGS. 15-16. The identifier may take various suitable forms, such as an ASCII code, sharable hyperlink, or an optical machine readable representation of data.

Continuing the example of FIG. 17, Dr. X can share the identifier 606, for example, with a second user, Dr. Y. Dr. Y then accesses the central server, for example via the internet, a local area network, or behind a firewall, depending on the particulars, to obtain the therapy pattern or package, as indicated at 608. For example, if Dr. Y is in the same clinical practice or hospital network as Dr. X, a private network may be established for therapy sharing, to prevent out-of-network users from accessing developed and possibly proprietary therapy patterns.

Dr. Y can then download the pattern or package and implement it on a patient, as indicated at 610. Depending on the particulars of a pattern or package, or patient, Dr. Y may tailor variables such as the amplitude/strength of the therapy and the electrodes to be used, for example. In some examples, the uploading step at 602 will allow Dr. X to designate those variables that may or may not be open for modification by the downloading user later on.

FIG. 18 illustrates another example method in block form. This method may be performed independently or may be inserted within FIG. 17 between blocks 600 and 602, or may be integrated as part of block 600 or 602 in FIG. 17. As noted at 630, Dr. X again has found a useful therapy for a condition, similar to FIGS. 16-17. Dr. X requests portability for the therapy pattern as indicated at 632. The CP, or Application, depending on the user context, then creates a package for the therapy pattern, as indicated at 634. For example, if the user, Dr. X, has generated the therapy pattern using a computer, tablet or other non-medical device, an Application may be used to package the therapy pattern for portability. If the user instead is operating the CP, or an RC, or other medical device, a utility to support portability may be part of the medical device programming. Once the therapy package is created, Dr. X can then share the therapy package, at indicated at 636.

Sharing 636 may take several forms. As indicated at 640, one approach is simply to share a file having the information necessary for reconstructing the therapy pattern and providing useful information for how to use the pattern, for example, including indications/condition for which the pattern may be useful, text relating to patient history, device implantation, or any other relevant information the originator (Dr. X here) chooses to include. File sharing 640 may occur, for example, by uploading the file to a shared folder on a local, shared server, or by sending the file as an attachment to an email, or by placing the file on a removable memory (thumb drive or SD card, for example) and physically handing the memory over, or via other file sharing services. In another example, the sharing 636 may include uploading the file to a remote or intranet-based server, which can in turn provide a link 642 that, when activated by clicking on it or copying and pasting into an internet browser, would take a recipient to the server for file download. In another example, an identifier or label may be generated, as indicated at 644, that may be used to search or pull up the relevant information for a therapy package from a server or library/repository for therapy packages.

In some illustrative embodiments, one or more data/safety checks may be added to the process. In a data check, the server would determine whether an appropriate file has been received using, for example, cyclic redundancy checking (CRC) or other data integrity check. The uploaded file may also be inspected to ensure it is not infected with a virus.

Safety checking may comprise determining whether a therapy pattern conforms to applicable design rules. Therapy patterns that are part of an uploaded therapy package may be subject to design rules, for example, limiting the total duration of an output, capping the duty cycle, or bounding the upper and/or lower limits of frequency content. Design rules may limit how many electrodes can be used at one time, or may limit current delivery or voltage differential between adjacent electrodes, for example. Design rules may allow or block the use of constant current and constant voltage therapy simultaneously. Design rules may address patient safety (such as a duty cycle or charge density limitation), or may address the actual hardware capabilities of a given system, or may be in selected to prevent the application of a therapy pattern that excessively uses energy or depletes battery capacity for an implantable system.

Data checking and design rules may be applied when a therapy pattern is to be encoded into a script as shown in FIGS. 15-16, when information is uploaded as in FIG. 17, or when a package is generated in FIG. 18, for example. Such rules may be again applied by a downloading device or system. In some examples, each of the uploading system, central server, and downloading system will perform data checking and design rule application. These rules may include the short term, intermediate term, and long-term management of the electrode interfaces described above.

In another example, therapy patterns may be shared after or as they are developed by, for example, machine learning algorithms. For example, an ambulatory patient is provided with a remote control (RC) to allow activation and tailoring of therapy. Machine learning algorithms can identify the activation and tailoring performed by the ambulatory patient via the RC and generate a therapy pattern that best matches the expressed wants of the ambulatory patient.

In a further example of machine learning, a closed loop system may observe, for example, the physiological response over time to a delivered therapy by capturing or identifying, for example, responses to therapy or the reemergence pathological signaling, and adjusting therapy output in response. As machine learning occurs, new therapy patterns may replace those originally programmed. Dr. X may obtain access to the pattern changes by remote (home) monitoring or at follow visits with the patient, and may share the new patterns developed over time.

It may be noted in these examples that a “therapy package” may define more than merely the set of outputs that a system may provide. In addition, the therapy package may define a response to sensed conditions or changes in sensed conditions such as, for example, calling for switching of electrodes for a given therapy output, or changing amplitude, in response to sensed conditions. Further, the therapy package may define plural patterns and changes to make over time to generate long term therapy approaches that account for the response of patient anatomy to therapy.

FIG. 19 shows an illustrative screenshot for a therapy pattern package. The screenshot 660 may be provided on any suitable display such as a computer screen or a screen on a mobile device, patient RC, or on a physician's CP. The screen may be a touchscreen.

The therapy pattern package is shown as having a name at 662 and an identifier at 664. The name 662 may be physician applied, while the identifier 664 may be an autogenerated code for use by a central server. In other examples, the name 662 may be for inputting the originating physician information. A field is provided for entering notes at 666, which may include, for example, a description of the patient condition in some detail, or patient age, gender and physical characteristics, or a description of implanted device position or other features. Notes may include clinical course information as well, or side effects or any other details that a user may wish to enter.

A list of conditions is shown at 668, for example, from Condition 1 to Condition N, for a selectable field. The individual conditions are listed at 670, with check boxes at 672 to identify any applicable patient conditions. Illustrative conditions may indicate a location of patient discomfort/pain, a particular type of patient disorder (pain, tremor, depression, or others, for example), and other suitable descriptors. If desired, a more open-ended approach may be taken to describing patient condition at 668, similar to the notes field at 666. It may be desirable in some examples to provide both open ended text entry and specific condition identifiers to facilitate field-based searching.

Therapy pattern information is provided at 674. This may include a graphical representation of components of a therapy output as indicated at 676A and 676B. A plurality of groups 678 may be identified, along with the type of output defined for each group (noted as a voltage output at 680A; a current controlled output is indicated by 680B. Electrode selection may be presented at 682, including whether the IPG canister (if the IPG is being used) is part of the electrode combination as shown at 684. In this example, separate electrode groups are identified for delivery of a synchronized voltage output 676A and current output 676B, with the current controlled therapy occurring during a quiet time in the voltage controlled therapy.

Though not shown, the originating user (physician or researcher) may also select which variables of a given therapy pattern can be modified by a subsequent user. For example, the originator may determine that some features, such as frequency or relative change in therapy amplitude, should be kept fixed, while others, such as the number of iterations of therapy to repeat, can be varied. In some embodiments, any variable may be selected and fixed; in other embodiments, only subsets of the available variables may be chosen.

The therapy package may be confirmed and saved by selecting block 690; if the therapy package is not presently open for editing, an edit block 692 is provided as well to allow the user to open up the therapy package for editing. To open a new therapy pattern package, a “New” button is provided at 694. One or more of blocks 690, 692, 694 may be omitted and/or unavailable at any given time when the screenshot 660 is being shown. For example, the edit block 692 would be greyed out during editing itself, while the confirm block 690 is active, and the edit block 692 would be available once the therapy package has been saved, with the confirm block 690 unavailable. Additional functions may be provided on the screen shot 660, or one or more of the elements shown may be available on a second screen—the set of items shown is merely illustrative for one example.

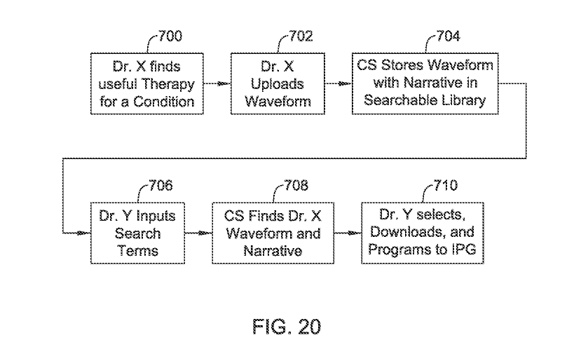

FIG. 20 shows another illustrative method in block form. In this example, Dr. X has again found a therapy useful for a condition at 700. Dr. X then uploads the waveform 702, for example as part of a therapy package that may comprise the waveform and associated information, such as a text narrative or other notes, labels, etc. as shown in FIG. 19. A central server then stores the therapy package in a searchable library 704. Dr. Y, wishing to treat a particular patient, inputs search terms 706 relevant to that patient's condition. The central server identifies waveforms that match the search terms 706, in block 708. Dr. Y is presented with one or more waveform patterns and/or packages to review, and selects and downloads the waveform package that Dr. Y thinks may work for the particular patient. Dr. Y then downloads the therapy pattern and programs an IPG, (or ETS, though not noted in the Figure) with the selected therapy pattern(s), as indicated at 710.

In some examples, Dr. X may be encouraged to participate in the method shown in FIG. 20 by providing remuneration. For example, Dr. X may receive payment when one or more steps takes place, including when Dr. X uploads the pattern/package at 702, or when Dr. Y's search turns up Dr. X's pattern/package at 708. In some examples, remuneration may wait until Dr. Y has actually implemented or programmed the therapy pattern/package to a patient's device. In further examples, an IPG or ETS is configured to count how often a given therapy pattern is used by a patient to treat a condition, and compensation may be tied to how frequently a therapy is delivered. Rather than directly compensating, for example on a per-use basis, it may be useful to instead use non-compensatory recognition, such as identifying those therapy patterns/packages that are most used and which physician originated the pattern.

In some examples, the searching capability at 706 may be limited to registered users for the CS and Searchable Library. In this way, access to therapy patterns and packages can be limited to those skilled in the art or who are authorized to practice medicine. In other examples, searching 706 may be allowed for any user, but downloading may be limited to registered users. In some examples, only registered or approved persons may upload at step 702 to the library. For example, only licensed practitioners or qualified researchers who have pre-registered may be allowed to upload therapy patterns.

In some examples, uploaders and/or users may be allowed to rate therapies which have been downloaded and tested, or may be allowed to post comments relating to the therapy, either privately (directed to the originator of the therapy pattern/package or to an administrator for the CS and/or Searchable Library) or for viewing by other users.

In some examples, in addition to or as an alternative to database searching, listing of therapy variables may be offered. In some examples, the search fields may include an originator or author search term to allow, for example, a user familiar with the work of Dr. X to search for patterns from Dr. X.

In some examples, a user (uploading, administering, or downloading) may tag a given file with literature reference(s) to the extent such references are relevant. For example, if Dr. X were to develop a therapy pattern and publish a paper or present a poster about the therapy pattern, the publication or poster may be linked or referenced with the therapy pattern/package.

In some examples, the user uploading a file may designate who is allowed to access the therapy pattern or package. For example, if Dr. X is a member of a clinical group or hospital network, Dr. X may designate that only other physicians in the group or network may search or download the therapy pattern.

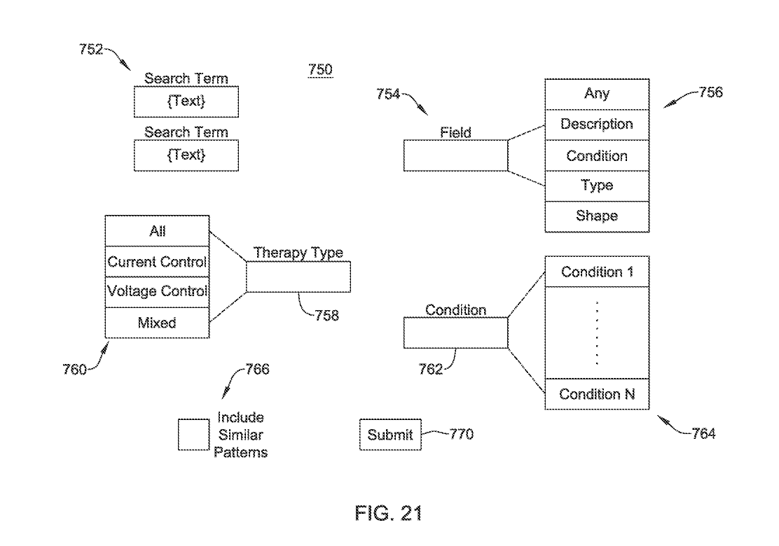

FIG. 21 shows an illustrative search screen. The Figure may show a screenshot at 750, though there are details provided that go beyond what the interface may directly include. At 752, the user would be allowed to enter text-based search terms freely. These terms may be compared to one or more fields, as indicated at 754. Fields, as indicated at 756, may include “Any”, or the description (such as data entered at 666 in FIG. 19), or the patient condition (such as data entered at 668 in FIG. 19), or the therapy type (which may include the voltage/current control notations 680A/680B in FIG. 19).

Shape is also included in the list at 756. “Shape” may represent a systematic classification of therapy patterns that can be extracted or provided as a characterization by the receiving server and/or the application software or medical device software that is used to create the therapy package. In some embodiments, when a therapy package is created or uploaded, the therapy pattern contained therein may be analyzed to identify similarities to existing therapy types, or to identify therapy waveform shape characteristics such as whether the therapy has particular shape content or frequency content. For example, the therapy pattern may be assessed via a Fourier transform to identify spectral content. In another example, the therapy pattern may be matched to predefined curves (ramps, square waves, sinusoidal or other curved patterns, for example) to characterize the type of content contained in a given pattern. Operations such as principle components analysis may be used, for example, to determine shape matching to pre-set pattern types. Using “Shape”, a physician looking for therapy having content with a selected frequency range, for example, 1-1.5 kHz, or selected shape type, for example, a sinusoidal, rather than square/rectangular wave, may narrow a search. The physician would indicate the desired shape, and the illustrative library system would search shape characterizations that have been performed on uploaded therapy patterns to identify those that contain the requested characteristic.

If desired, the search interface may also include drop-down menus for therapy type and/or condition. For example, a user may enter a therapy type at 758 selected from the drop down list shown at 760, including, All (which may be the default entry), Current Control, Voltage Control, or Mixed (that is, including both Current Controlled signals and Voltage Controlled signals in one pattern). The patient condition may also be selected from a drop-down list, using entry 762 and list 764. Alternatively, any of blocks 754, 758 and/or 762 can be free text search entries.

In an illustrative example, a checkbox can be provided as shown at 766 to allow the user to request that “similar patterns” to those that come up in the original search be included in the search results. This may address the situation where a physician looking to treat a given condition finds one or more patterns for that specific condition, but there may be other patterns not flagged for the selected patient condition that are morphologically similar to the search results. For example, pattern may be similar if it shows a 60% correlation (using, for example, correlation waveform analysis), or if a principal components analysis yields a match for the top principal component or at least two of the first to fourth principal components. Other standards, approaches and thresholds may be used to identify similarity.

By including the “similar” patterns in the results, a physician may be given the opportunity to explore additional therapy patterns, rather than being limited to only those already flagged for the condition searched.

Once the search terms are entered, the physician can hit the submit button 770. The system may then provide a search list, which may be ordered according to any of several methods. For example, the returned results may be sorted by those which most closely match the search criteria. The results may also be ordered by rating (if user ratings are allowed), or by the date of submission (newest or oldest first), or by number of users (if usage is tracked), or by any other suitable criteria. Search result sorting may be subject to preferences selected by the person requesting a search. Search results may be provided as plain text, or may include a brief description, a passage from a description, relevant conditions, one or more graphics showing the therapy patterns in a therapy, the name of the physician submitter or any other suitable component of a search record. In another example, search result sorting may done according to whether, how many, the caliber or, or the nature (whether peer reviewed, for example) of publications linked to a therapy package/pattern, where peer reviewed publication may be deemed of higher caliber, with other publications having lower priority, and therapy packages lacking publication links having the lowest priority.

While several references to sharing noted above refer to sharing from one physician to another physician, it should be noted that a physician may also share within the patients of a practice. For example, if two patients share similar conditions, a physician may infer that a new therapy pattern developed for a first patient may also work with a second patient. Therefore the patient may share from one patient to another by, for example, storing a therapy pattern on the CP, or on a thumb drive or mobile device, for reuse on a second patient.

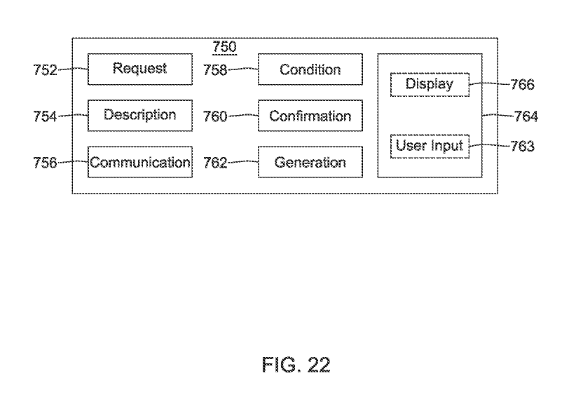

Referring to FIG. 22, the example shown may be a physician interface device 750 having a display 766 and a user input 768 and including operational circuitry for analyzing data, receiving user commands via the user input, and controlling the display to provide information to a user, the physician interface device configured to facilitate operation by a physician to package a therapy program the physician deems useful for sharing by operation of the following. The device 750 may include request means 752 to receive an input from a physician requesting that a selected therapy pattern be packaged for sharing.

The display 766 and user input 768 may both be provided as a touchscreen, with additional inputs, such as a keyboard, mousepad, etc., provided as additional inputs; the display 766 may also include a speaker and user input may include a microphone. The request means 752 may take the form of circuitry and/or program instructions for presenting an option to a physician user via the display 766 and obtaining an input indicating a request via the user input 768.

Upon receiving the request, the device 750 then uses condition means 758 to obtain/receive a user input to select one or more conditions to which the therapy pattern is applicable, where the condition means 758 may take the form of circuitry and/or program instructions to present a list of conditions that a physician may select (for example as shown at 668 in FIG. 19), or may allow free text entry, for example in a field that can be selected from the display 766. The device 750 may also use description means 754, which may comprise circuitry and/or program instructions to present a field for entry of descriptive text, or selecting descriptive terms (for example as shown at 666 in FIG. 19) to receive a user input to provide a description associated with the therapy pattern.

The device 750 may then use confirmation means 760, which may comprise circuitry and/or program instructions to present a selectable option (such as a button or a location on a touchscreen as shown at 690 in FIG. 19) to confirm that the therapy package is ready for sharing. The device 750 can then generate or construct a file or set of files using Generation block 762, which may comprise circuitry and or program instructions for storing in a file or package of files a combination of the set of data defining the therapy pattern and one or more sets of data to indicate any conditions to which the therapy pattern is applicable and any text received from the physician.

The device 750 may further include communication means, shown at 756, which may comprise circuitry and devices and/or programming instructions for communicating the package from the physician interface device to a second device. The communication means 756 may include, for example, transmission circuitry including an antenna for wireless communication, for example, such as cellular, Bluetooth, or WIFI communications, or other communication media and protocols, or may include a port for wired connection, such as to an Ethernet cable.

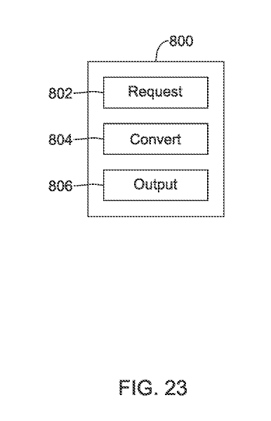

FIG. 23 shows another illustrative example. The example 800 includes receiving a request 802 to make a therapy pattern that has been developed a portable therapy package. The example, on receipt of a request 802, will then convert a therapy pattern to a therapy package that can be exported 806. The conversion at 804 may include requesting information from the physician using, for example, an interface as in FIG. 19. Alternatively, referring to FIG. 19, a guided process may be performed by allowing the user to go through individual screens for entering items in FIG. 19, which may include, for example, a name 662, identifier 664 (if the identifier is a user input), text 666 that may describe the therapy, theory behind the therapy, the type of patient target, considerations for tailoring a therapy pattern to a patient, or other elements desired by the person constructing the therapy package, as well as therapy conditions 668 to be flagged in association with the pattern, and as desired, any conditions of the therapy pattern that may be tailored by a subsequent user. In a guided process, the user may be allowed to observe the therapy pattern at each step, if desired.

The converted result, referring now again to FIG. 23 would be one or several files, or a .zip file, for example, that can be exported. In an example, there may be a data file and a .pdf file, where the data file is the pattern for export, in compressed form, if desired, while the .pdf file would contain images and information for example similar to what is shown in FIG. 19 (omitting blocks 692, 694, 696, as desired). The data and .pdf file would then be provided in a .zip package.

In one embodiment, the example may take the form of a method 800 comprising steps at 802, 804, 806. Alternatively, the example of FIG. 23 may be viewed as a representation of a device 800 having circuitry or programming instructions for operation by, for example, a controller or processor or circuits associated with such devices, that are configured for receiving a request 802, converting 804 a therapy pattern to a therapy package that can be exported or output 806 for use on a separate device.

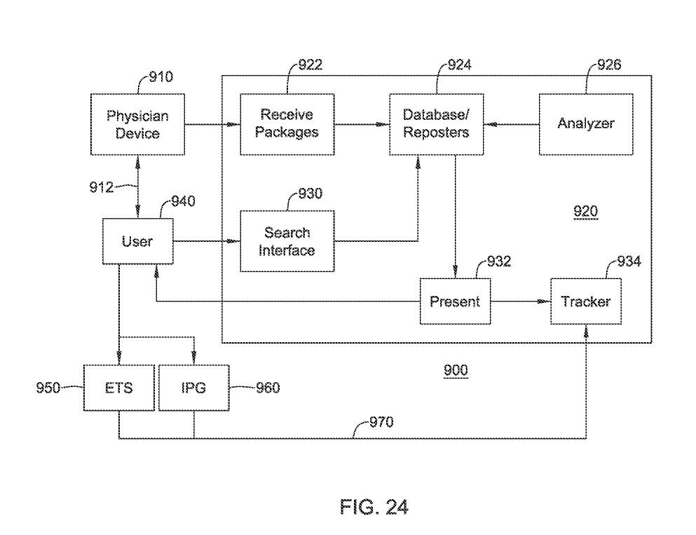

FIG. 24 shows a schematic system-level representation for an example. The example 900 shows interactions among one or more of a physician device 910, a user 940, an intermediary 920, and one or more of an external test system (ETS) 950 and implantable pulse generator (IPG) 960. The physician device 910 may be a clinician programmer (CP) as described above with reference to FIG. 1 or 22, or it may be physician's computer or mobile device having an application for developing and tailoring therapy patterns and converting to uploadable therapy package, as also noted relative to FIG. 22. The physician device 910 is used to develop a therapy package that is then uploaded to the intermediary 920.

The intermediary 920 may be one or several internet-connected servers, for example, or may be operated within a local or private network, if desired, for example in a clinical or hospital network.

The intermediary 920 includes circuitry and/or programming instructions for receiving a package 922. For example, in an internet protocol system, the intermediary 920 would receive a communicated message indicating that a therapy package is ready for loading, and the intermediary 920 would acknowledge the message and then receive the data stream comprising the file(s) for the therapy package. The therapy package would be loaded to a database or repository 924 which may be, for example, a memory location or locations for storing the therapy package and its components, which may be decompressed or unzipped, depending on the format of upload, if desired prior to storage. In some embodiments, the files are stored in compressed or zipped form, and may be decompressed or unzipped to facilitate analysis by an analyzer 926.

The analyzer 926 may comprise software and/or hardware (such as emulation circuitry) to allow for therapy pattern checking, such as operating the pattern in a simulated environment or in an emulation to determine the therapy pattern is suitable for use. Design rules may be applied, such as limitations on frequency, duty cycle, total amplitude or other limits suitable to the underlying ETS 950 or IPG 960 that will eventually be used to apply the pattern. In an example, analyzer 926, and/or the physician device 910 for uploading or the user downloading 940 may apply design rules related to correct maintenance of an electrode interface using the short term, intermediate term, and long term rulesets discussed above.

The analyzer 926 may also check for data integrity, virus scan, etc., to ensure that uploaded files are neither corrupt nor harmful. The analyzer 926 may also include software tools to characterize the therapy of a given therapy package or pattern, for example, identifying frequency components or shapes in the therapy to facilitate searching for specific pattern types (such as high frequency patterns, or patterns with sinusoidal, ramped or other specific therapy shaping). The analyzer 926 may perform signal deconstruction on a pattern to identify, for example, principal components thereof.

The analyzer 926 may still further include software tools for comparing an uploaded therapy pattern in a package against other already loaded patterns/packages to identify similarity. Similarity may be used to provide additional searching results in response to a query as discussed above relative to FIG. 21 at 766. Similarity may also be used to help determine compensation decisions to prevent, for example, copying of an uploaded therapy pattern/package from one physician by a second physician if a compensation scheme is in place to reward those physicians who come up with novel therapy patterns that other users implement. Outcomes from the analyzer 926 may be used to determine which records go into the database 924, as well as providing additional data and tags on the records provided in the database.

A user 940 may use a CP or his or her own computer to input searches or other queries to the intermediary 920 via a search interface 930 using, for example, an interface as shown above in FIG. 21. The search interface 930 passes on relevant parts of the query to the database 924, and results are then presented via 932. Block 932 may provide a simple list of result, or may include various informational fields such as graphic representation of the therapy pattern, identification of entered text or identified conditions in the therapy package, metrics or assessments provided by the analyzer, etc. The presentation of a given therapy package may be tracked as well, as indicated at 934, for counting or compensation purposes.

The user 940 may then select from among the presented 932 therapy packages those which can be tested for a patient using the ETS 950, or which may be implemented by the IPG 960. As indicated at 970, which therapy packages/patterns are used may also be tracked by the tracker 934 of the intermediary 920, for example, for counting or compensation or other reward purposes. One ultimate outcome may be that the most used patterns, may become preloaded or default patterns for use in an ETS 950 or IPG 960, or may be preloaded on CP devices.

In one example, the user 940 may rely on communication 912 from the originating physician to indicate a code or identifier of a specific pattern, rather than searching using, for example, condition descriptions or therapy type information to search. Such an approach may address a regulatory environment that may view search tools such as those described herein based on text or treatable condition as one or more of the provision of medical services or off-label use if a therapy pattern is provided to be implemented by a user 940 in response to patient condition-based search terms. Using the code or identifier provided by a second physician may avoid any perception of medical practice being performed by the intermediary 920, and should also avoid any off-label use allegation.

As regards the pattern description, in some examples the specific electrodes for use in delivering a particular therapy may vary from one patient to another based on patient condition and the placement of electrodes in the patient relative to target anatomy. For these examples, the specific electrode selection may be omitted from pattern description, but what may be included is a set of timing information for sets of electrodes to deliver therapy. That is, in an example, the implanting physician may tailor or select sets of electrodes as befits a given patient but the therapy pattern may dictate the order, timing, or spatial patterning in which electrodes become active for voltage or current output.

In another example, electric field related data may be provided relative to a specific location where a physician using a pattern may enter, for example, the distance from a target structure to a lead structures (such as a paddle lead), as well as the relative placement thereof, and a pattern may direct the selection of electrodes and relative amplitudes of therapeutic output from such electrodes. This patient specific information may be entered by the user 940 and either the user block 940 or the intermediary may automatically generate a therapy pattern for implementation from the entered user information.

The user 940 may operate in the system shown by use of a CP, which can automatically convert a therapy package with a desired pattern to a set of output parameters using known characteristics of a lead and/or electrode placement. For example, the user 940 may obtain information regarding the characteristics of the IPG 960 or ETS 950, and/or the lead(s) being used therewith, or the location of implantation of such leads. These details can then be used to give effect to a desirable pattern using a stored therapy package from the intermediary 920. For example, knowing the characteristics of a paddle lead, which can be identified by a part number or other product identifier, the spatial patterning of a therapy output would be known if, for example, a distance to a target structure is also known. For a larger paddle with wider spaced electrodes, a desirable pattern may take a first form, while a smaller paddle with closer spaced electrodes may require a different form for the same pattern. In another example, imaging or impedance information may be used to determine spacing and juxtaposition of electrodes relative to anatomy and/or one another, for entry into a CP.

In the following non-limiting examples, various means for performing certain functions are described with reference to block diagrams above. It should be understood, as noted above, that such blocks may represent dedicated circuitry/hardware, stored instruction sets for execution by a processor or controller, and/or combinations thereof. Certain of these non-limiting examples may be implemented in a CP or a physician or researcher laptop, mobile device, tablet, smartphone, etc.

A first non-limiting example takes the form of a physician interface device having a display and a user input and including operational circuitry for analyzing data, receiving user commands via the user input, and controlling the display to provide information to a user, the physician interface device configured to facilitate operation by a physician to package a therapy program the physician deems useful for sharing by operation of the following: request means to receive an input from a physician requesting that a selected therapy pattern be packaged for sharing; confirmation means to confirm that the therapy package is ready for sharing; and communication means for communicating the package from the physician interface device to a second device. FIG. 22 shows an example of a physician interface device having a user input 763 and display 766 (which may be combined as a touch screen), which is described as including operational circuitry comprising a request block 752, a confirmation block 760, and communication block 756. FIG. 19 shows an example for a screen for interacting with the physician having overall the user input and display (shown at 660) with a block for requesting a new input (“New”, 694), and a block for confirming therapy package (“Confirm”, at 690).

A second non-limiting example takes the form of a physician interface device as in the first non-limiting example, further comprising condition means to receive a user input to select one or more conditions to which the therapy pattern is applicable; and description means to receive a user input to provide a description associated with the therapy pattern. FIG. 22 further shows a physician interface device having a block 758 for the patient condition, as well as a description block 754 for receiving a description of the therapy pattern. FIG. 19 shows an interface example with a portion for receiving patient condition information at 668, and a notes block 666 for describing the pattern.

A third non-limiting example takes the form of a physician interface device as in the second non-limiting example, wherein the display is configured such that at least one of the request means, condition means, description means, and confirmation means, is displayed alongside a graphical representation of at least a portion of the therapy pattern. FIG. 19 illustrates placement of the request, condition, description and confirmation blocks alongside graphical representation of patterns at 674.

A fourth non-limiting example takes the form of a physician interface device as in any of the first to third non-limiting examples, wherein the display and user input are provided as a combined unit in the form of a touchscreen having visual output and tactile input. FIGS. 19 and 22 both indicate touchscreen usage. A fifth non-limiting example takes the form of a physician interface device as in any of the first to fourth non-limiting examples, wherein the means for communicating comprises one or more of a connection jack for a communication cable, or an antenna and associated circuitry for delivering a wireless output. A sixth non-limiting example takes the form of a physician interface device as in any of the first to fourth non-limiting examples, wherein the means for communicating is configured to deliver the package to a central server adapted for receiving therapy pattern packages.

A seventh non-limiting example takes the form of a physician interface device configured to allow a physician to tailor therapy for a patient using a therapy pattern, wherein the device comprises at least a display for displaying information to a user and a user input for receiving inputs form a user, and operational circuitry configured to control the display and receive inputs from the user input, the operational circuitry comprising: request means for allowing a user to request that a therapy pattern be reduced to a sharable script; conversion means for converting the therapy pattern to a sharable script; and output means to provide a user with the sharable script. FIG. 23 shows an illustrative example, in which a device 800 comprises a request block 802 for receiving the user request, a conversion block 804 for converting a therapy pattern into a sharable script, and an output block 806 for providing the output.

An eighth non-limiting example takes the form of a physician interface device as in the seventh non-limiting example, wherein the operational circuitry is further configured to receive a script for at therapy pattern from a physician and convert the script to a therapy pattern for provision to a therapy delivery device. For example, the conversion block 804 may perform an operation to convert the entered script back to a therapy pattern. A ninth non-limiting example takes the form of a device as in either of the seventh or eighth non-limiting examples wherein the script is a text based code comprising one or more of letters and numbers in a specific sequence. Examples of such scripts are noted in FIG. 15 overall as well as at block 554 in FIG. 16. A tenth non-limiting example takes the form of a device as in either of the seventh or eighth non-limiting examples, wherein the script is provided as an optical machine readable data representation. An example of such a script is noted above in block 554 of FIG. 16.

An eleventh non-limiting example takes the form of a method of providing a therapy library for access by medical providers to obtain therapy patterns for neurological therapy, the method comprising: receiving one or more therapy packages from one or more physician interface devices (for example, FIG. 24 at block 922), in which therapy packages comprise an identifier of a condition the therapy is useful for, a description, and a pattern definition (such elements of a therapy package are illustrated in the screen shot of FIG. 19); providing a search interface to a search user (for example, FIG. 24, at block 930), the search interface allowing a user to select or enter one or more of: a pattern description, a patient condition, or patient description (such elements of a search interface are shown in FIG. 21); receiving a search request from a search user (user 940 interacts with the search interface 930 in FIG. 24); identifying one or more therapy packages matching one or more elements of the search request; (the search interface 930 interacts with the database 924) and presenting to the search user at least one therapy package (an output is presented via block 932 to the user 940).

A twelfth non-limiting example takes the form of a method of facilitating patient treatment by physicians comprising providing an interface allowing a user to enter one or more of patient characteristics or therapy characteristics as a search request (search interface 930 in FIG. 24 may operate according to the search interface shown in FIG. 21); receiving a search request (as shown in FIG. 24, search interface 930 allows a user input from user 940); searching a database of therapy packages, (the search interface 930 interacts with the database 924 in FIG. 24) the therapy packages including descriptions and therapy patterns for electrical therapy delivery (therapy packages may be defined according to one or several of the fields illustrated in FIG. 19); identifying one or more therapy packages matching the search request as results and providing a list of results to the user (conclusion of operations by the search interface 930 on the database 924 operates via the present block 932 to output data to the user 940); receiving an input from the user selecting a therapy package from the list of results (continued interaction between the presentation block 932 and the user 940); and communicating therapy package data to the user comprising a set of therapy parameters for use in a medical device under the control of the user (as described above, block 940 represents both the user as an individual and, in some examples, a clinician programmer or remote control, and receives the therapy package including a therapy pattern from the presentation block 932 and delivers to a selected one or both of the ETS 950 and/or IPG 960).

A thirteenth non-limiting example takes the form of a method as in the twelfth non-limiting example, wherein the step of providing the interface is performed on a user interface of a clinician programmer for a neuromodulation device. A fourteenth non-limiting example takes the form of a method as in the twelfth or thirteenth non-limiting examples, wherein the step of searching the database comprises accessing an searching a database located remotely from the clinician programmer.

A fifteenth non-limiting example takes the form of a medical device system configured to assess a proposed therapy delivery pattern against a set of rules, the therapy delivery pattern comprising a sequence of electrical therapy outputs for delivery via electrodes in contact with patient tissue, the set of rules comprising: a first rule setting one or more upper limits on therapeutic output via the electrodes; a second rule setting a charge burden limit defined by a quantity of charge on an electrode for a period of time; and a third rule calling for long term zeroing of charge on each electrode interface; wherein the medical device system comprises at least one of: a programmer for providing instructions to an implantable medical device; or a computing system for receiving data remotely from one or more implantable medical devices and/or programmers; and the medical device system comprises: receiving means for receiving a therapy package comprising a candidate therapy delivery pattern; and analyzer means for emulating or simulating the candidate therapy delivery pattern and determining whether each of the first, second and third rules are met. Such a system is illustratively shown in FIG. 24 in which the analyzer 926, or, alternatively, the physician device 910 and/or user device 940, apply such short term, intermediate term, and long term rulesets to protect the electrode-tissue interface.

A sixteenth non-limiting example takes the form of a physician interface device configured to allow a physician to tailor therapy for a patient using a therapy pattern, wherein the device comprises at least a display for displaying information to a user and a user input for receiving inputs form a user, and operational circuitry configured to control the display and receive inputs from the user input, the operational circuitry being configured to perform the following: receiving an input from a physician requesting that a selected therapy pattern be packaged for sharing; combining the selected therapy pattern and one or more of patient information or description as a therapy package; confirming that the therapy package is ready for sharing; and communicating the package from the physician interface device to a second device.

A seventeenth non-limiting example takes the form of a physician interface device as in the sixteenth non-limiting example, wherein the operational circuitry is further configured for:

receiving a user input to select one or more conditions to which the therapy pattern is applicable; and receiving a user input to provide a description associated with the therapy pattern. An eighteenth non-limiting example takes the form of a physician interface device as in the seventeenth non-limiting example, wherein the display is configured to display separate fields for each of: receiving the user request; selecting the one or more conditions; providing the description associated with the therapy pattern; and a graphical representation of at least a portion of the therapy pattern.

A nineteenth non-limiting example takes the form of a physician interface device as in the sixteenth non-limiting example wherein the display and user input are provided as a combined unit in the form of a touchscreen having visual output and tactile input. A twentieth non-limiting example takes the form of a physician interface device as in the sixteenth non-limiting example further comprising one or more of a connection jack for a communication cable, or an antenna and associated circuitry for delivering a wireless communication output. A twenty-first non-limiting example takes the form of a physician interface device as in the sixteenth non-limiting example wherein the step of communicating the package is performed by sending the package electronically to a central server adapted for receiving therapy pattern packages.

A twenty-second non-limiting example takes the form of a physician interface device configured to allow a physician to tailor therapy for a patient using a therapy pattern, wherein the device comprises at least a display for displaying information to a user and a user input for receiving inputs form a user, and operational circuitry configured to control the display and receive inputs from the user input, the operational circuitry being configured to perform the following: providing a prompt to a user to request that a therapy pattern be reduced to a sharable script; receiving a request from a user and converting the therapy pattern to a sharable script; and providing a user with the sharable script.

A twenty-third non-limiting example takes the form of a physician interface device as in the twenty-second non-limiting example wherein the operational circuitry is further configured to receive a script for at therapy pattern from a physician and convert the script to a therapy pattern for provision to a therapy delivery device. A twenty-fourth non-limiting example takes the form of a physician interface device as in the twenty-second non-limiting example, wherein the script is a text based code comprising one or more of letters and numbers in a specific sequence. A twenty-fifth non-limiting example takes the form of a physician interface device as in the twenty-second non-limiting example, wherein the script is provided as an optical machine readable data representation.

A twenty-sixth non-limiting example takes the form of a method of providing a therapy library for access by medical providers to obtain therapy patterns for neurological therapy, the method comprising: receiving one or more therapy packages from a physician interface device, in which therapy packages comprise an identifier of a condition the therapy is useful for, a description, and a pattern definition; providing a search interface to a search user, the search interface allowing a user to select or enter one or more of: a pattern description, a patient condition, or patient description; receiving a search request from a search user; identifying one or more therapy packages matching one or more elements of the search request; and presenting to the search user at least one therapy package.

A twenty-seventh non-limiting example takes the form of a method as in the twenty-sixth non-limiting example, wherein the presenting step includes presenting the at least one therapy package in a prioritized order. A twenty-eighth non-limiting example takes the form of a method as in the twenty-sixth non-limiting example, further comprising identifying one or more therapy packages that are similar to the at least one therapy package that is presented, wherein similarity is determined by assessment of one or more of correlation analysis, frequency content or principal components analysis on a plurality of stored therapy packages, and presenting to the search user at least one similar therapy package. A twenty-ninth non-limiting example takes the form of a method as in the twenty-sixth non-limiting example, wherein the presenting step comprises presenting a graphical representation of a therapy pattern for a presented therapy package.

A thirtieth non-limiting example takes the form of a method of facilitating patient treatment by physicians comprising: providing a prompt on a user interface allowing a user to enter one or more of patient characteristics or therapy characteristics as a search request; receiving a search request; searching a database of therapy packages, the therapy packages including descriptions and therapy patterns for electrical therapy delivery; identifying one or more therapy packages matching the search request as results; and providing a list of results to the user via the user interface.

A thirty-first non-limiting example takes the form of a method as in the thirtieth non-limiting example, further comprising: receiving an input from the user selecting a therapy package from the list of results; and communicating therapy package data to the user comprising a set of therapy parameters for use in a medical device under the control of the user. A thirty-second non-limiting example takes the form of a method as in the thirty-first non-limiting example, wherein the step of providing the interface is performed on a user interface of a clinician programmer for a neuromodulation device. A thirty-third non-limiting example takes the form of a method as in any of the thirtieth to thirty-second non-limiting examples, wherein the step of searching the database comprises accessing and searching a database located remotely from the clinician programmer. A thirty-fourth non-limiting example takes the form of a method as in any of the thirtieth to thirty-third non-limiting examples, further comprising identifying one or more therapy packages that are similar to the at least one therapy package that is presented as a result, wherein similarity is determined by assessment of one or more of correlation analysis, frequency content or principal components analysis on a plurality of stored therapy packages, and presenting to the search user at least one similar therapy package as one of the results.

A thirty-fifth non-limiting example takes the form of a medical device system configured to assess a proposed therapy delivery pattern against a set of rules, the therapy delivery pattern comprising a sequence of electrical therapy outputs for delivery via electrodes in contact with patient tissue, the set of rules comprising: a first rule setting one or more upper limits on therapeutic output via the electrodes; a second rule setting a charge burden limit defined by a quantity of charge on an electrode for a period of time; and a third rule calling for long term zeroing of charge on each electrode interface; wherein the medical device system includes operational circuitry configured to: receive a therapy package comprising a candidate therapy delivery pattern; emulate or simulate the candidate therapy delivery pattern; determine whether each of the first, second and third rules are met; and find that the second rule is not met, determine that the proposed therapy delivery pattern is inappropriate for use with a given implantable therapy system and prevent sharing of the proposed therapy delivery pattern.

A thirty-sixth non-limiting example takes the form of a medical device system as in the thirty-fifth non-limiting example, wherein the system takes the form of a clinician programmer with a user interface for receiving instructions from a clinical user and communication circuitry for communicating with an implantable or external medical device configured to deliver electrical therapy for neurological treatment.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A physician interface device configured to allow a physician to tailor therapy for a patient using a therapy pattern, wherein the physician interface device comprises at least a display for displaying information to a user and a user input for receiving inputs from a user, and operational circuitry configured to control the display and receive inputs from the user input, the operational circuitry being configured to perform the following:
receiving an input from a physician requesting that a selected therapy pattern be packaged for sharing;
combining the selected therapy pattern and one or more of patient information or description as a therapy package;
confirming that the therapy package is ready for sharing; and
communicating the therapy package from the physician interface device to another device.

2. The physician interface device of claim 1 wherein the operational circuitry is further configured for:
receiving a user input to select one or more conditions to which the therapy pattern is applicable; and
receiving a user input to provide a description associated with the therapy pattern.

3. The physician interface device of claim 2 wherein the operational circuitry is further configured to control the display to show separate fields for each of:
receiving the user request;
selecting the one or more conditions;
providing the description associated with the therapy pattern; and
a graphical representation of at least a portion of the therapy pattern.

4. The physician interface device of claim 1, wherein the display and user input are provided as a combined unit in the form of a touchscreen having visual output and tactile input.

5. The physician interface device of claim 1 further comprising one or more of a connection jack for a communication cable, or an antenna and associated circuitry for delivering a wireless communication output.

6. The physician interface device of claim 1 wherein the step of communicating the therapy package is performed by sending the therapy package electronically to a central server adapted for receiving therapy packages.

7. A method of providing a therapy library for access by medical providers to obtain therapy patterns for neurological therapy, the method comprising:
receiving one or more therapy packages from a physician interface device, in which therapy packages comprise an identifier of a condition the therapy is useful for, a description, and a pattern definition;
providing a search interface to a search user, the search interface allowing a user to select or enter one or more of: a pattern description, a patient condition, or patient description;
receiving a search request from a search user;
identifying one or more therapy packages matching one or more elements of the search request; and
presenting to the search user at least one therapy package.

8. The method of claim 7 wherein the presenting step includes presenting the at least one therapy package in a prioritized order.

9. The method of claim 7 further comprising identifying one or more therapy packages that are similar to the at least one therapy package that is presented, wherein similarity is determined by assessment of one or more of correlation analysis, frequency content or principal components analysis on a plurality of stored therapy packages, and presenting to the search user at least one similar therapy package.

10. The method of claim 7 wherein the presenting step comprises presenting a graphical representation of a therapy pattern for a presented therapy package.

11. The method of claim 7 further comprising:
validating the identify of a search user; and
allowing a validated search user to download a therapy package.

12. The method of claim 11 wherein validating the identity of the search user is performed by determining that the search user is a physician authorized to download therapy packages.

13. The method of claim 7 further comprising, upon receiving a therapy package, analyzing the therapy package to determine that it meets one or more safety rules.

14. The method of claim 13 wherein the one or more safety rules comprise rules for management of an electrode interface of an implantable medical device.

15. The method of claim 13 wherein the one or more safety rules comprise:
a first rule setting one or more upper limits on therapeutic output via the electrodes;
a second rule setting a charge burden limit defined by a quantity of charge on an electrode for a period of time; and
a third rule calling for long term zeroing of charge on each electrode interface;

16. A method of facilitating patient treatment by physicians comprising:
providing a prompt on a user interface allowing a user to enter one or more of patient characteristics or therapy characteristics as a search request;
receiving a search request;
searching a database of therapy packages, the therapy packages including descriptions and therapy patterns for electrical therapy delivery;
identifying one or more therapy packages matching the search request as results; and
providing a list of results to the user via the user interface.

17. The method of claim 16 further comprising:
receiving an input from the user selecting a therapy package from the list of results; and
communicating therapy package data to the user comprising a set of therapy parameters for use in a medical device under the control of the user.

18. The method of claim 17 wherein the step of providing the interface is performed on a user interface of a clinician programmer for an neuromodulation device.

19. The method of claim 17 wherein the step of searching the database comprises accessing and searching a database located remotely from the clinician programmer.

20. The method of claim 17 further comprising identifying one or more therapy packages that are similar to the at least one therapy package that is presented as a result, wherein similarity is determined by assessment of one or more of correlation analysis, frequency content or principal components analysis on a plurality of stored therapy packages, and presenting to the search user at least one similar therapy package as one of the results.
